SEQUENCE LISTING (15 sequences)

SEQ ID NO: 1 - DNA, 40 bp, Artificial Sequence
  chemically-synthesized capture probe
aaaaaaaccccagcaattcaagtgtgttgagagctttgat

SEQ ID NO: 2 - DNA, 25 bp, Artificial Sequence
  chemically-synthesized capture probe
aaaaaattgagagctttgattcgtg

SEQ ID NO: 3 - DNA, 37 bp, Artificial Sequence
  chemically-synthesized PCR hybridization probe
atcaaagctctcaacgcctgcaagtcctaagacgcca

SEQ ID NO: 4 - DNA, 45 bp, Artificial Sequence
  chemically-synthesized hybridization probe
gttacttcgttcgattgtctggacttataatgctgaacttctggt

SEQ ID NO: 5 - DNA, 41 bp, Artificial Sequence
  chemically-synthesized hybridization probe
cttcgttcgattgtctggacttataatgctgaacttctggt

SEQ ID NO: 6 - DNA, 39 bp, Artificial Sequence
  chemically-synthesized hybridization probe
tcgttcgattgtctggacttataatgctgaacttctggt

SEQ ID NO: 7 - DNA, 19 bp, Artificial Sequence
  chemically-synthesized PCR primer
catggcaccaccagcatct

SEQ ID NO: 8 - DNA, 20 bp, Artificial Sequence
  chemically-synthesized PCR primer
atccgcgtgtttcttttcga

SEQ ID NO: 9 - DNA, 42 bp, Artificial Sequence
  chemically-synthesized cleaved tag
cacgaatcaaagctctcaacgcctgcaagtcctaagacgcca

SEQ ID NO: 10 - DNA, 45 bp, Artificial Sequence
  chemically-synthesized capture probe
gtgcttagtttcgagagttgtgtgaacttaacgaccccaaaaaaa

SEQ ID NO: 11 - DNA, 42 bp, Artificial Sequence; identical to SEQ ID NO: 9
  chemically-synthesized cleaved tag

SEQ ID NO: 12 - DNA, 25 bp, Artificial Sequence
  chemically-synthesized capture probe
aaaaaagtgcttagtttcgagagtt

SEQ ID NO: 13 - DNA, 37 bp, Artificial Sequence; identical to SEQ ID NO: 3
  chemically-synthesized cleaved tag

SEQ ID NO: 14 - DNA, 25 bp, Artificial Sequence
  chemically-synthesized capture probe
aaaaaagtgcttagtttcgagagtt

SEQ ID NO: 15 - DNA, 23 bp, Artificial Sequence
  chemically-synthesized target-specific segment
cgcctgcaagtcctaagacgcca

(19) United States
(12) Patent Application Publication
(10) Pub. No.: US 2008/0241838 A1
(43) Pub. Date: Oct. 2, 2008

(54) METHODS AND SYSTEMS FOR DETECTING NUCLEIC ACIDS

(75) Inventors: Kristian Scaboo, Castro Valley, CA (US); Vissarion Aivazachvili, Oakland, CA (US); Timothy Liu, Fremont, CA (US); Robert Eason, Los Gatos, CA (US); Konrad Faulstich, Salem (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: APPLERA CORPORATION, APPLIED BIOSYSTEMS GROUP, Foster City, CA (US)

(21) Appl. No.: 11/965,837

(22) Filed: Dec. 28, 2007

Related U.S. Application Data
(60) Provisional application No. 60/880,964, filed on Jan. 18, 2007.
(60) Provisional application No. 60/880,428, filed on Jan. 16, 2007.
(60) Provisional application No. 60/877,610, filed on Dec. 29, 2006.

(51) Int. Cl. C12Q 1/68

(52) U.S. Cl. 435/6

(57) ABSTRACT

Methods and kits for detecting a target nucleic acid in a sample are described. In some embodiments, the sample to be analyzed includes a primer which hybridizes to at least a portion of the target nucleic acid, a probe having a first region which hybridizes to at least a portion of the target nucleic acid and a second region having a detectable label, a polymerase which extends the hybridized primer and an enzyme comprising nuclease activity that can cleave the hybridized hybridization probe to thereby release a labeled probe fragment. In some embodiments, the sample can then be contacted with a solid support comprising surface bound capture probes which can hybridize to the labeled probe fragment(s). These capture probes more readily bind to the probe fragment(s) than to the intact hybridization probe. The label can then be detected on the support surface. In this manner, improved discrimination between the probe fragments and the intact hybridization probes can be achieved.

This application claims the benefit of Provisional U.S. Patent Application Nos.: 60/877,610, filed on Dec. 29, 2006; 60/880,428, filed on Jan. 16, 2007; and 60/880,964, filed on Jan. 18, 2007. Each of these applications is incorporated by reference herein in its entirety.

Pursuant to the provisions of 37 C.F.R. § 1.52(e)(5), the sequence listing text file named 0045USU1_ST25, created on Dec. 27, 2007 and having a size of 3,544 bytes, and which is being submitted herewith, is incorporated by reference herein in its entirety.

The section headings used herein are for organizational purposes only and should not be construed as limiting the subject matter described herein in any way.

### FIELD

This application relates generally to systems and methods for detecting biological molecules and, in particular, to systems and methods for detecting nucleic acids in a sample.

### INTRODUCTION

Nucleic acid amplification may be performed in conjunction with a variety of assays. Such assays may be qualitative, for example when used to evaluate a biological sample. However, a wide variety of biological applications could be improved by the ability to detect the amplification of target nucleic acids, without requiring either cumbersome blotting techniques, or the expensive and delicate equipment typically required for optical methods.

Accordingly, there still exists a need for improved methods for detecting nucleic acids in a sample.

### SUMMARY

According to a first embodiment, a method of detecting a target nucleic acid in a sample is provided which comprises:

incubating the sample with:
a primer which hybridizes to at least a portion of the target nucleic acid;
a hybridization probe comprising first and second regions, wherein the first region hybridizes to at least a portion of the target nucleic acid and the second region does not hybridize to the target nucleic acid, the second region comprising a detectable label; and
a polymerase and an enzyme comprising a nuclease activity wherein the polymerase extends the hybridized primer in the direction of the hybridized probe and the nuclease activity of the enzyme cleaves the hybridized probe to thereby release a probe fragment comprising the second region and the detectable label;

allowing the primer and the hybridization probe to hybridize to target nucleic acid in the sample;

allowing the polymerase to extend the hybridized primer;

allowing the nuclease activity of the enzyme to cleave the hybridized hybridization probe to thereby release the probe fragment;

contacting the sample with a surface of a solid support, wherein the surface of the solid support comprises one or more capture probes each of which hybridizes to at least a portion of the second region of the probe fragment(s);

allowing the capture probes to hybridize to probe fragment(s) in the sample to form a probe fragment/capture probe complex; and

detecting the label on the surface of the solid support;

wherein the capture probe more readily binds to the probe fragment than to the intact hybridization probe and wherein the hybridization probe is substantially single stranded at the Tₘ of the probe fragment/capture probe complex.

According to a second embodiment, a method for detecting a target nucleic in a sample is provided which comprises:

melting the sample by heating the sample to a first temperature, wherein the sample comprises:

a primer which hybridizes to at least a portion of the target nucleic acid;

a hybridization probe comprising first and second regions, wherein the first region hybridizes to at least a portion of the target nucleic acid and the second region does not hybridize to the target nucleic acid and wherein the second region comprises a detectable label; and

a polymerase and an enzyme comprising nuclease activity wherein the polymerase extends the hybridized primer in the direction of the hybridized probe and the nuclease activity of the enzyme cleaves the hybridized probe to thereby release a probe fragment comprising the second region of the probe and the detectable label; and

and wherein the first temperature is above the melting temperature (Tₘ) of the primer and double stranded nucleic acids present in the sample;

subsequently annealing the sample by reducing the temperature to a second temperature lower than the first temperature to allow the primer and the hybridization probe to each hybridize to a single stranded portion of the target nucleic acid in the sample; and

subsequently elongating the primer by allowing the polymerase to extend the primer hybridized to the target nucleic acid at a third temperature thereby releasing the probe fragment;

optionally repeating melting, annealing and elongating at least once;

contacting the sample with a surface of a solid support, wherein the surface of the solid support comprises one or more capture probes which hybridize to at least a portion of the second region of the probe fragment;

allowing the capture probe to hybridize to at least a portion of the probe fragments in the sample to form a probe fragment/capture probe complex at a fourth temperature lower than the second and third temperatures; and

detecting label on the surface of the solid support;

wherein the capture probe more readily binds to the probe fragment than to the intact hybridization probe and wherein the hybridization probe is substantially single stranded at the Tₘ of the probe fragment/capture probe complex.

According to a third embodiment, a kit for detecting a target nucleic acid in a sample is provided which comprises:

a hybridization probe comprising a first region which hybridizes to at least a portion of the target nucleic acid and a second region comprising a detectable label wherein the second region does not hybridize to the target nucleic acid and wherein an enzyme comprising nuclease activity can cleave the hybridization probe when hybridized to the target nucleic acid to thereby produce a probe fragment comprising the second region and the detectable label;

a solid support comprising one or more capture probes on a surface thereof, wherein the capture probes can hybridize to at least a portion of the second region of the probe fragment to form a probe fragment/capture probe complex and wherein the capture probe more readily binds to the probe fragment than to the intact hybridization probe and wherein the hybridization probe is substantially single stranded at the Tₘ of the probe fragment/capture probe complex;

optionally, a primer which hybridizes to at least a portion of the target nucleic acid; and

optionally, a polymerase which extends the hybridized primer in the direction of the hybridized probe and an enzyme comprising nuclease activity to thereby cleave the hybridized hybridization probe and release the probe fragment comprising the second region of the probe and the detectable label.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a photograph of a multiplexed chip which can be used to detect nucleic acids in a sample.

FIG. 2 is a graph showing square wave voltammograms which illustrate the discrimination between positive samples and no template controls (NTC's) for Capture Probe 1, which has 25 bases between the hybridization region and the electrode surface, and Capture Probe 2, which has only 6 bases between the hybridization region and the electrode surface.

FIG. 3 is a bar graph of the electrochemical signal for the multiplexed chip modified with either Capture Probe 2 (3 runs) or Capture Probe 1 (2 runs) showing the improved discriminating capabilities of Capture Probe 2.

FIGS. 4A and 4B are schematic depictions illustrating the hybridization of cleaved and uncleaved probes to Capture Probe 1 (FIG. 4A) and Capture Probe 2 (FIG. 4B) illustrating the enhanced discrimination effect of Capture Probe 2.

FIGS. 5A-5C are bar graphs showing the results for hybridization of 19 mer, 15 mer and 13 mer hybridization probe fragments, respectively, to a 20 mer capture probe.

FIG. 6 is a schematic depiction of a electrochemical cell having a gold working electrode (WE) and a platinum counter electrode (CE).

FIG. 7 is a bar graph showing the signal generated for hybridization of three different hybridization probe/probe fragment combinations.

FIGS. 8A-8C are schematic depictions showing binding of the hybridization probe to the capture probe for the combinations used in FIG. 7.

FIG. 9 is a depiction of a scheme for the synthesis of an osmium complexing agent that can be coupled to the 5′ amino group of a probe to form an Os-labeled probe.

### DETAILED DESCRIPTION

For the purposes of interpreting of this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” herein means “and/or” unless stated otherwise or where the use of “and/or” is clearly inappropriate. The use of “a” herein means “one or more” unless stated otherwise or where the use of “one or more” is clearly inappropriate. The use of “comprise,” “comprises,” “comprising” “include,” “includes,” and “including” are interchangeable and not intended to be limiting. Furthermore, where the description of one or more embodiments uses the term “comprising,” those skilled in the art would understand that in some specific instances, the embodiment or embodiments can be alternatively described using language “consisting essentially of” and/or “consisting of.”

As used herein, “capture probe” refers to a nucleobase polymer that is surface bound. The capture probe can be a nucleic acid (e.g. DNA or RNA), a nucleic acid analog (e.g. locked nucleic acid (LNA)), a nucleic acid mimic (e.g. peptide nucleic acid (PNA)) or a chimera.

As used herein, “chimera” refers to a nucleobase polymer comprising two or more linked subunits that are selected from different classes of subunits. For example, a PNA/DNA chimera would comprise at least one PNA subunit linked to at least one 2′-deoxyribonucleic acid subunit (For exemplary methods and compositions related to PNA/DNA chimera preparation See: WO96/40709). Exemplary component subunits of a chimera are selected from the group consisting of PNA subunits, naturally occurring amino acid subunits, DNA subunits, RNA subunits, LNA subunits and subunits of other analogues or mimics of nucleic acids.

As used herein, “flap” refers to a portion of a hybridization probe that is non-complementary to the target nucleic acid the probe is designed to determine.

As used herein, “hybridization probe” is a nucleobase polymer that can be cleaved by nuclease activity of an enzyme at a site where the probe is hybridized to a complementary strand, said hybridization probe comprising a nucleobase sequence that is complementary to at least a portion of a target nucleic acid of interest in a sample. The hybridization probe can be a oligonucleotide, oligonucleotide analog or chimera so long as it is cleavable by nuclease activity. In some embodiments, the nucleobase polymer can be a chimera that comprises all DNA subunits except for one LNA subunit. In some embodiments, the nucleobase polymer comprises a single LNA subunit that is situated one subunit removed (toward the 3′ end) from the 5′ end of that portion of the hybridization probe that is designed to hybridize to the target nucleic acid.

As used herein, “nuclease activity” refers to the ability of an enzyme to cleave the backbone of a nucleobase polymer (e.g., a nucleic acid). Non-limiting examples of nuclease activity include exonuclease activity (i.e., the ability of an enzyme to cleave nucleotide sequences sequentially from the free end of a nucleobase polymer substrate) and endonuclease activity (i.e., the ability of a protein to recognize specific, short sequences of a nucleobase polymer and to cleave the nucleobase polymer at those sites).

As used herein, “nucleobase polymer” refers to a polymer comprising a series of linked nucleobase containing subunits. Non-limiting examples of suitable polymers include oligodeoxynucleotides, oligoribonucleotides, peptide nucleic acids, nucleic acid analogs, nucleic acid mimics and chimeras.

As used herein, “peptide nucleic acid” or “PNA” refers to any polynucleobase strand or segment of a polynucleobase strand comprising two or more PNA subunits, including, but not limited to, any polynucleobase strand or segment of a polynucleobase strand referred to or claimed as a peptide nucleic acid in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 and 6,357,163. For the avoidance of any doubt, PNA is a nucleic acid mimic and not a nucleic acid or nucleic acid analog. PNA is not a nucleic acid since it is not formed from nucleotides. For the avoidance of doubt, PNA oligomers may include polymers that comprise one or more amino acid side chains linked to the backbone.

As used herein, “support”, “solid support” or “solid carrier” refers to any solid phase material. Solid support encompasses terms such as “resin”, “synthesis support”, “solid phase”, “surface” “membrane” and/or “support”. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled-pore-glass (CPG), or reverse-phase silica. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, tube, channel, cylinder or other container, vessel, feature or location.

As used herein, “target nucleic acid” refers to a nucleic acid molecule of interest. A sample can comprise more than one target nucleic acid molecule.

Methods for electrochemically monitoring the outcome of a PCR reaction are disclosed in U.S. patent application Ser. No. 11/488,439, filed on Jul. 17, 2006. This application describes assays which employ a hybridization probe which, upon cleavage, yields a cleavage product that is a single-stranded oligonucleotide that can hybridize at a modified electrode surface for detection. This type of detection involves separation of the cleaved probe from the uncleaved probe. Separation, for example, can be accomplished by capture of a biotin labeled probe on a streptavidin matrix.

The present inventors have discovered that the ability to discriminate between cleaved and intact hybridization probes can also be achieved without separation by modifying the structure of the solid phase capture probe and/or the hybridization probe. In particular, it has been discovered that the structure of the solid phase capture probe and/or the hybridization probe has an affect on the ability of the capture probe to discriminate between the intact hybridization probe and the cleaved probe fragment. While not wishing to be bound by theory, it is believed that this phenomenon results from a steric hindrance effect that inhibits hybridization of the intact or uncleaved (i.e., the longer or more bulky) hybridization probe to the capture probe.

According to one embodiment, the hybridization probe is substantially single stranded at the Tₘ of the probe fragment/capture probe complex wherein “substantially single stranded” means that less than 5% of the hybridization probe is part of a double stranded complex (e.g., a folded structure).

### Electrode Surface Capture Probes

Two electrode capture probe sequences were investigated for their ability to discriminate between the cleaved hybridization probe fragment and the intact hybridization probe. The capture probe sequences employed in the following experiments differ by the distance of nineteen bases between the hybridization region, which is shown in boldface and underlined below, and the gold surface. The sequences of the two capture probes are shown below, with the portion of the sequence homologous to the second region of the probe fragment(s) produced by cleavage of the hybridization probe shown in bold and underlined.

Capture Probe 1:

(SEQ ID NO: 1)

5′ (DTPA)(DTPA)(DTPA) AAA AAA ACC CCA GCA ATT CAA
GTG TGT TGA GAG CTT TGA T 3′
Capture Probe 2:

(SEQ ID NO: 2)

5′ (DTPA)(DTPA)(DTPA) AAA AAA TTG AGA GCT TTG ATT
CGT G 3′

The oligonucleotides were modified on the 5′ end with the dithiol phosphoramidite (DTPA) (Glen Research, Inc) for attachment to the gold electrodes. The capture probes were attached to the electrodes using the following procedure. First, the electrodes were cleaned by exposure to an UV Ozone Cleaner (Jelight Inc) for 20 min followed by an ethanol soak to reduce the oxide formed. Then, 40 μL of a 1 uM solution of the thiolated capture probe in 1M Phosphate buffer (pH 7) was deposited on the surface for 15 min in an electrode area defined by a silicone well (Molecular Probes, Inc). The electrodes were then rinsed in water and exposed to a 2 mM mercaptohexanol solution for 2 hrs. After exposure, the electrodes were rinsed in water and dried under argon.

### PCR Hybridization Probe

The PCR hybridization probe was obtained from IDT Inc. with a 5′ amine modification so that it could be coupled in-house to an electroactive Ferrocene (Fc) moiety. The sequence is as follows with the 5′ flap indicated in bold and underlined:

(SEQ ID NO: 3)

5′ Fc-ATC AAA GCT CTC AAC GCC TGC AAG TCC TAA GAC
GCC A-biotin

The biotin modification on the 3′ end was not utilized in the following experiments.

### PCR Conditions For Listeria

PCR was performed in 1× buffer A from core PCR kit (Applied Biosystems Ca# N808-0228) supplemented with 6 mM MgCl₂. PCR primers and Ferrocene labeled hybridization probe were present at concentrations 200 nM and 400 nM, respectively. The 25 μL reaction mix contained either 3000 or 0 copies of Listeria DNA for positive and negative (i.e., no template control) samples. Cycling parameters were as follows: 95° C. for 10 min., then (95° C. for 15 sec and 66° C. for 30 sec)×40 cycles. Immediately after PCR, the 25 μL reaction mix was placed on the gold electrode and covered with a glass coverslip for 1 hr static hybridization at room temperature. Alternatively, the PCR mix was introduced into the multiplexed electrode chip for flow-through detection.

### Multiplexed Electrode Chip

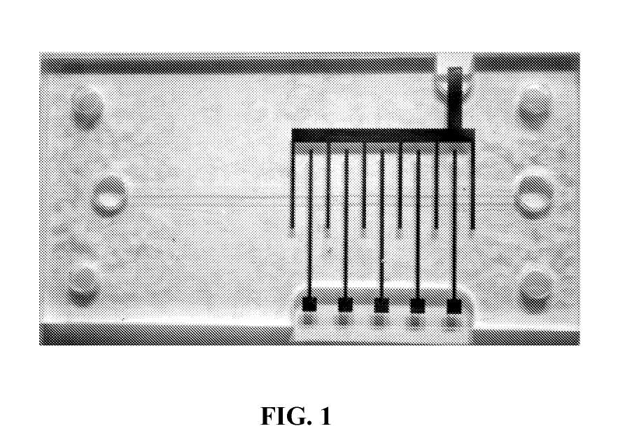

FIG. 1 is a photograph of a multiplexing chip which can be used for electrochemical measurements. As shown in FIG. 1, the chip includes 500 μm wide gold finger working electrodes inter-digitated with a pronged counter electrode crossing a flow channel of 350 μm width and 120 μm height. The gold electrodes are modified before assembly with the indicated capture probes and then the chip is assembled. Subsequently, 20 μL of the completed PCR solutions are flowed through the chip at a flow rate of 1 μL/min to allow for hybridization.

All electrochemical measurements are performed as described previously.

### Results

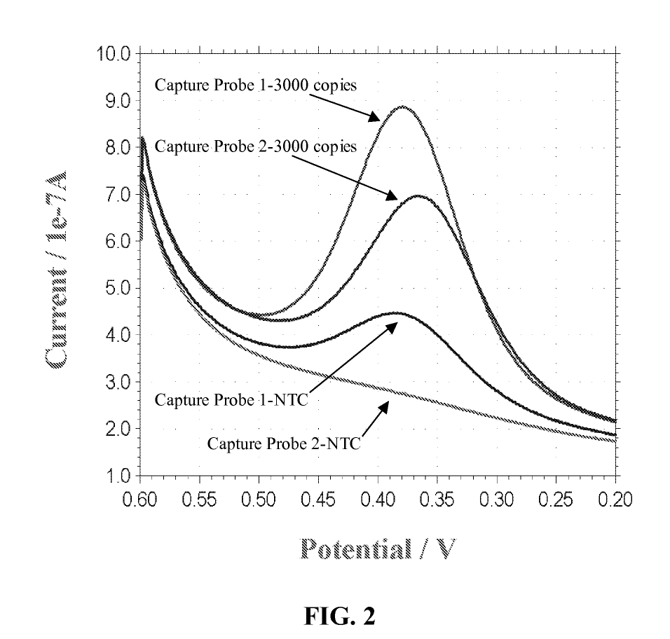

FIG. 2 shows the results for the planar gold electrode with a static, 1 hour hybridization. As can be seen from the data in FIG. 2, discrimination between the positive sample and the no template control (NTC) can be seen for both capture probes. However for Capture Probe 2, there is much lower signal from the NTC sample. This indicates that the intact hybridization probe does not hybridize well to that probe surface.

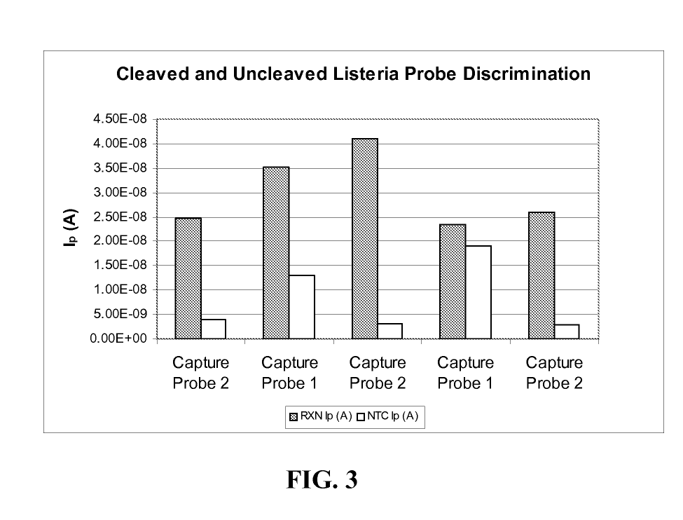

The results obtained using the multiplexed, flow through chip of FIG. 1 are set forth in FIG. 3. The data depicted in FIG. 3 is summarized in the table below.

| | | | | | Dis. |
|---|---|---|---|---|---|
| | RXN | | NIC | | Ratio |
| | Iₚ (A) | A (VA) | Iₚ (A) | A (VA) | Rxn/NTC |
| Capture | 2.48E−08 | 2.40E−09 | 3.90E−09 | 4.02E−10 | 6.4 |
| Probe 2 | | | | | |
| Capture | 3.51E−08 | 3.57E−09 | 1.30E−08 | 1.14E−09 | 2.7 |
| Probe 1 | | | | | |
| Capture | 4.11E−08 | 4.20E−09 | 3.08E−09 | 3.40E−10 | 13.3 |
| Probe 2 | | | | | |
| Capture | 2.34E−08 | 2.25E−09 | 1.90E−08 | 1.76E−09 | 1.2 |
| Probe 1 | | | | | |
| Capture | 2.60E−08 | 2.71E−09 | 2.94E−09 | 2.90E−10 | 8.8 |
| Probe 2 | | | | | |

wherein “Dis. Ratio” represents the discrimination ratio of the capture probes.

For purposes of the present application, the “discrimination ratio” of the capture probe is the ratio obtained by dividing the intensity of the signal generated from the positive sample by the intensity of the signal generated by the no template control (NTC) under the conditions and using the protocol set forth above.

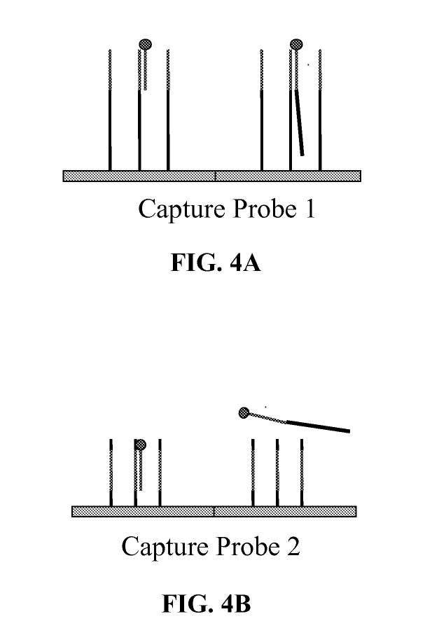

While not wishing to be bound by any theory, it is believed that the shorter capture probe which has a hybridization region closer to the surface of the electrode prevents efficient hybridization of the full length, intact hybridization probe to the capture probe. This steric hindrance effect is illustrated in FIGS. 4A and 4B. In particular, FIG. 4A illustrates the hybridization of cleaved and intact hybridization probes to the longer Capture Probe 1 and FIG. 4B illustrates the hybridization of cleaved and intact hybridization probes to the shorter Capture Probe 2.

The ability of the capture probe to discriminate between the probe fragment and the intact hybridization probe can also be enhanced by variations in said hybridization probe. In particular, the present inventors have discovered that the length of the 5′ flap of the hybridization probe (which 5′ flap is part of the probe fragment after cleavage of the hybridization probe by the nuclease activity of the enzyme) also influences the ability of the capture probe to discriminate between cleaved and intact hybridization probe.

To demonstrate this phenomenon, a bird flu assay was conducted using the following hybridization probes:

19-mer 5′ flap

(SEQ ID NO: 4)

GTTACTTCGTTCGATTGTC^{▾}TGGACTTATAATGCTGAACTTCTGGT
15-mer 5′ flap

(SEQ ID NO: 5)

CTTCGTTCGATTGTC^{▾}TGGACTTATAATGCTGAACTTCTGGT
13-mer 5′ flap

(SEQ ID NO: 6)

TCGTTCGATTGTC^{▾}TGGACTTATAATGCTGAACTTCTGGT

The nucleobases illustrated above in bold in these sequences represent the 5′ flap and the ^{▾} symbol represents the site where cleavage by the exonucleoase activity is expected to be predominant. In these hybridization probes, the underlined C nucleobase that is adjacent to the illustrated cleavage site is an LNA subunit. All other subunits of these hybridization probes are DNA.

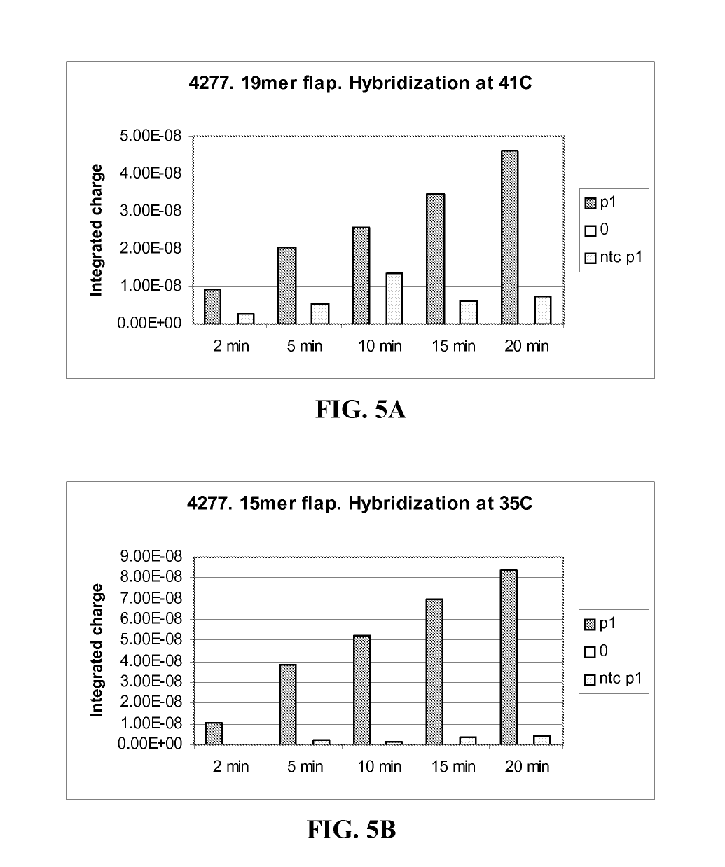

When cleaved by the nuclease activity of the enzyme, the probe of SEQ ID NO: 4 produces, when cleaved, a probe fragment comprising the 19 mer 5′ flap whereas the probe of SEQ ID NO: 5 produces a probe fragment comprising the 15 mer 5′ flap and the probe of SEQ ID NO: 6 produces a probe fragment comprising the 13 mer 5′ flap. Bar graphs showing the results for hybridization to a 20 mer capture probe are provided in FIGS. 5A-5C for the each of the probe fragments comprising the 19 mer, 15 mer and 13 mer 5′ flaps, respectively. As can be seen from these charts, the probe fragment comprising the 13 mer flap produces much higher discrimination between the cleaved and intact hybridization probe than do the probe fragments comprising longer flaps. Thus, it seems that shortening of the 5′ flap decreases the binding of the intact hybridization probe to the surface bound capture probe. For the data in FIGS. 5A-5C, hybridizations were carried out at temperatures 10° C. below the predicted Tₘ of the duplexes formed by the second region (i.e. the nucleobase sequence of the 5′ flap) of the probe fragments and the capture probe.

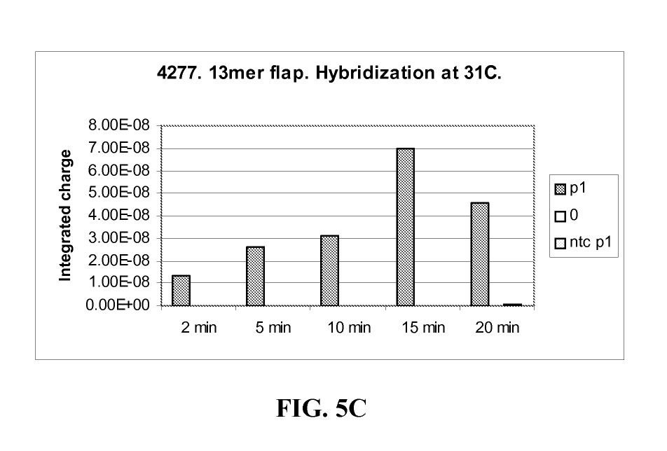

FIG. 6 is a schematic depiction of an electrochemical cell having a gold working electrode (WE) and a platinum counter electrode (CE) that can be used in the above described assays. As shown in FIG. 6, the electrochemical cell is formed by sandwiching a PDMS gasket between the counter and working electrodes. The working electrode (WE) and the counter electrode (CE) can have diameters of 2 mm. The platinum counter-electrode (CE) can be made by sputter coating a 2000 Angstrom thick platinum layer on a silicon wafer having a Cr adhesion layer. The gold counter-electrode (CE) can be made by sputter coating a 2000 Angstrom thick gold layer on a silicon wafer having a Cr adhesion layer. The reference electrode can be a 0.5 mm diameter Ag/AgCl wire.

### Detection of Cleaved Tag In the Presence of Uncleaved Probe

This example illustrates embodiments in which a tag complement is immobilized on an electrode by thiol moieties (here provided by DTPA moieties) that exhibit specificity for binding to gold surfaces, such as a gold electrode, and a cleavable probe that contains (i) a polynucleotide sequence attached to the 5′ end of a target complementary segment and (ii) a detectable tag comprising an osmium-containing complex for electrochemical detection after capture of the cleaved tag by the immobilized tag complement.

The cleaved probe can be detected and/or measured in the presence of uncleaved probe by selection of an appropriate capture probe (a tag complement) such that the capture probe destabilizes capture of uncleaved (intact) probe by selectively binding the tag of the uncleaved probe close to the electrode surface. As a result, the capture probe hybridizes to the cleaved tag more stably than the uncleaved tag moiety bound to the probe.

A 50 μl reaction mix is prepared that contains 1×PCR buffer A (Applied Biosystems, P/N N808-0228), 6 mM MgCl₂, 200 μM of each dNTP, 200 nM of forward and reverse primers, 400 nM 5′-Os-labeled probe, 0.05 units of Gold AmpliTaq™ polymerase and 3,000 copies of Listeria monocytogenesis DNA.

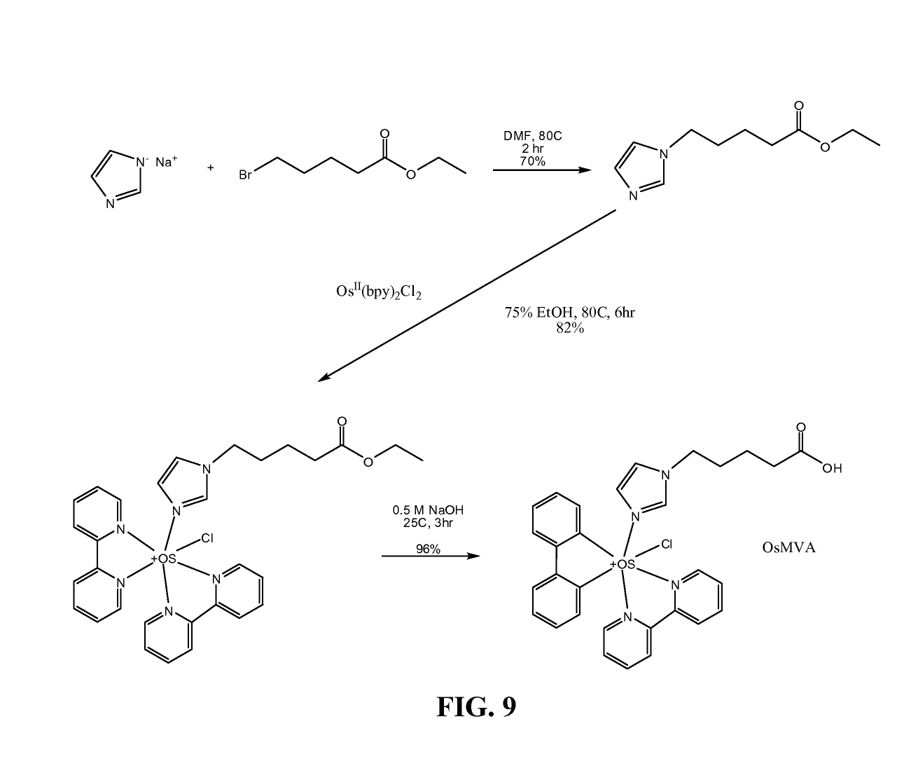

The osmium complex labeling agent that was coupled to the 5′ amino group of each probe to form the Os-labeled probe is shown in FIG. 9 along with a scheme for the synthesis of the osmium complexing agent. The forward and reverse primers used during the PCR were as follows:

| | |
|---|---|
| 5′-CATGGCACCACCAGCATCT | SEQ ID NO: 7 |
| and | |
| 5′-ATCCGCGTGTTTCTTTTCGA | SEQ ID NO: 8 |

Three different combinations of cleavable probes and immobilized tag complements were tested, as shown in the following combinations in which the upper sequence (underlined) represents the Os-labeled cleaved tag to be detected, and the lower sequence represents a capture probe that was attached to the electrode by 3 DTPA moieties at its 5′ end, and contained a tag complement for binding to the tag sequence:

Combination #1
Tag 1:

SEQ ID NO: 9

5′-CACGAATCAAAGCTCTCAAX-3′
Cap1:

SEQ ID NO: 10

3′-GTGCTTAGTTTCGAGAGTTGTGTGAACTTAACGACCCCAAAAAAA5′
Combination #2
Tag 1:

SEQ ID NO: 11

5′-CACGAATCAAAGCTCTCAAX-3′
Cap2:

SEQ ID NO: 12

3′-AAAAAAGTGCTTAGTTTCGAGAGTT(C18)5′
Combination #3
Tag 2:

SEQ ID NO: 13

5′-ATCAAAGCTCTCAAX-3′
Cap2:

SEQ ID NO: 14

3′ AAAAAAGTGCTTAGTTTCGAGAGTT(C18)5′

wherein X is:

SEQ ID NO: 15

CGCCTGCAAGTCCTAAGACGCCA-3′ (target-specific
segment)
and
C18 is (OCH₂CH₂)₆(DTPA)₃

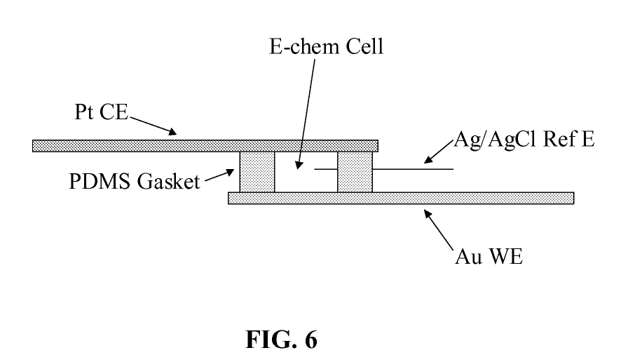

Thermocycling was performed at 95° C. for 10 min., then (92° C. for 15 sec, 66° C. for 30 sec.)×40 cycles. Then, the PCR mix was loaded into an electrochemical cell of the type depicted in FIG. 6 for electrochemical measurements. The measurements were performed using a 1 M NaCl hybridization buffer at 31 ° C. (which is approximately 10 degrees below the melt temperature (Tₘ) of the 15-mer cleaved tag sequence in Combination #3 above as calculated using the Tₘ calculator program on IDT web site: www.idt.com). Results are shown in FIG. 7.

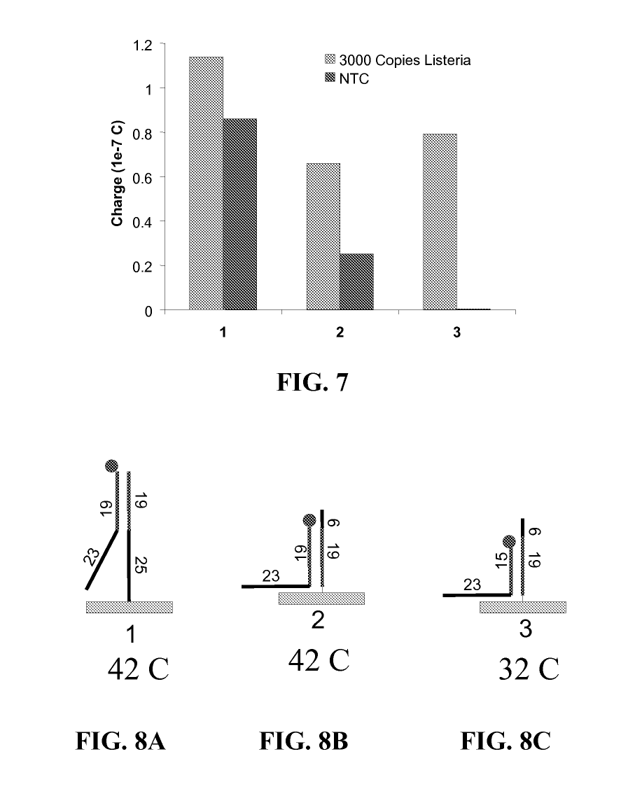

FIG. 7 is a bar graph showing the results for hybridization of the three different hybridization probe/probe fragment combinations set forth above. A schematic depiction of the binding of the intact hybridization probes to the capture probe for each of the combinations is shown in FIGS. 8A, 8B and 8C. As can be seen from FIGS. 8A-8C, each hybridization probe had a 23 mer region which did not hybridize to the capture probe. The hybridization probes used in Combinations 1 and 2 also had a 19 mer region (i.e. a 5′ flap) that hybridized to the capture probe whereas the hybridization probe used in Combination 3 had a shorter 15 mer region (i.e. 5′ flap) that hybridized to the capture probe. The capture probe used in Combination 1 had a 25 mer spacer region between the support surface and the region that hybridizes to the hybridization probe. In contrast, the capture probes used in Combinations 2 and 3 did not have the 25 mer spacer region between the support surface and the region that hybridizes to the 5′ flap of the hybridization probe. As can be seen from FIG. 7, Combination 3 provided by far the highest level of discrimination between the cleaved hybridization probe fragment and the intact hybridization probe. The hybridization probe used in Combination 3 had the shortest region which hybridized to the capture probe (15 mers). For Combinations 1 and 2, hybridization to the capture probe was conducted at 42° C. whereas for Combination 3, hybridization was conducted to 32° C.

As set forth above, the ability of the capture probe to discriminate between the probe fragment and the intact hybridization probe can be enhanced by variations in both the sequence and length of the capture probe as well as by modifications of the hybridization probe. The hybridization probe can also be modified by extending the 3′ end of the hybridization probe or by adding a bulky modification on the 3′ end of the hybridization probe that would further block access to the capture probe sequence on the solid support surface.

Any known electrochemical moiety can be used as a label on the cleaved portion of the hybridization probe. Exemplary electrochemical labels which may be used include bis(2,2′-bipyridyl)imidizolylchloroosmium(II) [salt]. This label gives a good Eₒ of 0.165 vs Ag/AgCl and has good solubility properties for synthesis and purification. Other exemplary labels include ferrocene as well as the labels disclosed in U.S. patent application Ser. No. 11/488,439 filed on Jul. 17, 2006. Moreover, the electrochemical label can be any moiety that can transfer electrons to or from an electrode. Exemplary electrochemical labels include transition metal complexes. Suitable transition metal complexes include, for example, ruthenium²⁺ (2,2′-bipyridine)₃ (Ru(bpy)₃²⁺), ruthenium²⁺(4,4′-dimethyl-2,2′-bipyridine)₃ (Ru(Me²-bpy)₃²⁺), ruthenium²⁺(5,6-dimethyl-1,10-phenanthroline)₃ (Ru(Me₂-phen)₃²⁺), iron2+(2,2′-bipyridine)₃ (Fe(bpy)₃²⁺), iron²⁺(5-chlorophenanthroline)₃ (Fe(5-Cl-phen)₃²⁺), osmium²⁺(5-chlorophenanthroline)₃ (Os(5-Cl-phen)₃²⁺), osmium²⁺(2,2′-bipyridine)₂ (imidazolyl), dioxorhenium¹⁺ phosphine, and dioxorhenium¹⁺ pyridine (ReO₂ (py)₄¹⁺). Some anionic complexes useful as mediators are: Ru(bpy)((SO₃)₂-bpy)₂²⁻ and Ru(bpy)((CO₂)₂-bpy)₂²⁻ and some zwitterionic complexes useful as mediators are Ru(bpy)₂ ((SO₃)₂-bpy) and Ru(bpy)₂((CO₂)₂-bpy) where (SO₃)₂-bpy₂- is 4,4′-disulfonato-2,2′-bipyridine and (CO₂)₂-bpy₂- is 4,4′-dicarboxy-2,2′-bipyridine. Suitable substituted derivatives of the pyridine, bypyridine and phenanthroline groups may also be employed in complexes with any of the foregoing metals. Suitable substituted derivatives include but are not limited to 4-aminopyridine, 4-dimethylpyridine, 4-acetylpyridine, 4-nitropyridine, 4,4′-diamino-2,2′-bipyridine, 5,5′-diamino-2,2′-bipyridine, 6,6′-diamino-2,2′-bipyridine, 4,4′-diethylenediamine-2,2′-bipyridine, 5,5′-diethylenediamine-2,2′-bipyridine, 6,6′-diethylenediamine-2,2′-bipyridine, 4,4′-dihydroxyl-2,2′-bipyridine, 5,5′-dihydroxyl-2,2′-bipyridine, 6,6′-dihydroxyl-2,2′-bipyridine, 4,4′,4″-triamino-2,2′,2″-terpyridine, 4,4′,4″-triethylenediamine-2,2′,2″-terpyridine, 4,4′,4″-trihydroxy-2,2′,2′-terpyridine, 4,4′,4″-trinitro-2,2′,2″-terpyridine, 4,4′,4″-triphenyl-2,2′,2″-terpyridine, 4,7-diamino-1,10-phenanthroline, 3,8-diamino-1,10-phenanthroline, 4,7-diethylenediamine-1,10-phenanthroline, 3,8-diethylenediamine-1,10-phen anthroline, 4,7-dihydroxyl-1,10-phenanthroline, 3,8-dihydroxyl-1,10-phenanthroline, 4,7-dinitro-1,10-phenanthroline, 3,8-dinitro-1,10-phenanthroline, 4,7-diphenyl-1,10-phenanthroline, 3,8-diphenyl-1,10-phenanthroline, 4,7-disperamine-1,10-phenanthroline, 3,8-disperamine-1,10-phenanthroline, dipyrido[3,2-a:2′,2′-c]phenazine, and 6,6′-dichloro-2,2′-bipyridine, among others.

In addition, although electrochemical detection is exemplified above, the disclosed methods are also applicable to the detection of nucleic acids by other detection techniques, such as fluorescence detection. Moreover, the detectable label on the hybridization probe can be any moiety which is capable of being detected and/or quantitated. Exemplary labels include electrochemical, luminescent (e.g., fluorescent, luminescent, or chemiluminescent) and calorimetric labels.

The primers and probes used herein may have any of a variety of lengths and configurations. For example, the primers may be from 18 to about 30 subunits in length or from 20 to 25 subunits in length. Longer or shorter length primers can also be used. The length of the region of the hybridization probe which binds to the target nucleic acid can be from 8 to 30 subunits whereas the length of the region of the hybridization probe which does not bind to the target can have a length of 2 to 40 subunits or from 8 to 30 subunits. Hybridization probes having longer or shorter regions than those exemplified above can also be used.

The primers (e.g., PCR primers) may be designed to bind to and produce an amplified product of any desired length, usually at least 30 or at least 50 nucleotides in length and up to 200, 300, 500, 1000, or more nucleotides in length. The probes and primers may be provided at any suitable concentrations. For example, forward and reverse primers for PCR may be provided at concentrations typically less than or equal to 500 nM, such as from 20 nM to 500 nm, or 50 to 500 nM, or from 100 to 500 nM, or from 50 to 200 nM. Probes are typically provided at concentrations of less than or equal to 1000 nM, such as from 20 nM to 500 nm, or 50 to 500 nM, or from 100 to 500 nM, or from 50 to 200 nM. Exemplary conditions for concentrations of NTPs, enzyme, primers and probes can also be found in U.S. Pat. No. 5,538,848 (hereby incorporated by reference), or can be achieved using commercially available reaction components (e.g., as can be obtained from Applied Biosystems, Foster City, Calif.).

A plurality of complementary capture probes, each having a characteristic sequence, may also be used. For example, an array of capture oligonucleotides that hybridize to different hybridization probe fragments may be used to localize and capture individual tag sequences in a plurality of discrete detection zones.

The methods described herein can be used to detect target nucleic acid in real time. For example, the solid support can be in contact with the solution in which nucleic acid amplification is occurring and the process monitored during PCR (i.e. real-time detection). Alternatively, detection of probe fragments can also be conducted after the amplification process is complete (i.e., end-point detection). In some embodiments, the PCR assay can be monitored during PCR (real-time) and after the process is completed (i.e. end-point). PCR assays can be performed using traditional PCR formats as well as Fast PCR formats, asymmetric PCR formats and asynchronous PCR formats.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method of detecting a target nucleic acid in a sample, the method comprising:
incubating the sample with:
a primer which hybridizes to at least a portion of the target nucleic acid;
a hybridization probe comprising first and second regions, wherein the first region hybridizes to at least a portion of the target nucleic acid and the second region does not hybridize to the target nucleic acid, the second region comprising a detectable label; and
a polymerase and an enzyme comprising an nuclease activity wherein the polymerase extends the hybridized primer in the direction of the hybridized probe and the nuclease activity of the enzyme cleaves the hybridized probe to thereby release a probe fragment comprising the second region and the detectable label;
allowing the primer and the hybridization probe to hybridize to target nucleic acid in the sample;
allowing the polymerase to extend the hybridized primer;
allowing the nuclease activity of the enzyme to cleave the hybridized hybridization probe to thereby release the probe fragment;
contacting the sample with a surface of a solid support, wherein the surface of the solid support comprises one or more capture probes each of which hybridizes to at least a portion of the second region of the probe fragment;
allowing the capture probes to hybridize to probe fragment in the sample to form a probe fragment/capture probe complex; and
detecting the label on the surface of the solid support;
wherein the capture probe more readily binds to the probe fragment than to the intact hybridization probe and wherein the hybridization probe is substantially single stranded at the Tₘ of the probe fragment/capture probe complex.

2. The method of claim 1, wherein the capture probe has a discrimination ratio of 3 or greater.

3. The method of claim 1, wherein the capture probe has a discrimination ratio of 5 or greater.

4. The method of claim 1, wherein the second region of the probe fragment binds to the capture probe such that the portion of the second region adjacent the first region in the intact hybridization probe is oriented toward the solid support surface.

5. The method of claim 4, wherein a proximal region of the capture probe adjacent to the solid support surface does not hybridize to the probe fragment and a distal region of the capture probe away from the solid support surface hybridizes to the second region of the probe fragment.

6. The method of claim 5, wherein the proximal region of the capture probe is shorter than the first region of the hybridization probe.

7. The method of claim 5, wherein the first region of the hybridization probe comprises a moiety which inhibits the binding of the intact hybridization probe to the capture probe via steric hindrance.

8. The method of claim 7, wherein the capture probe and the hybridization probe each comprise polynucleotides.

9. The method of claim 8, wherein the proximal region of the capture probe has fewer nucleotides than the first region of the intact hybridization probe.

10. The method of claim 8, wherein the polynucleotides comprise deoxyribonucleotides.

11. The method of claim 1, wherein the polymerase and the enzyme comprising a nuclease activity are the same molecule.

12. The method of claim 11, wherein the polymerase is a thermostable enzyme.

13. The method of claim 12, wherein the thermostable enzyme is Taq polymerase.

14. The method of claim 1, wherein the surface of the solid support comprises an electrode and wherein the detectable label is a moiety that can transfer electrons to or from the electrode.

15. The method of claim 14, wherein the detectable label is a Ferrocene moiety.

16. The method of claim 14, wherein the surface of the solid support comprises gold.

17. The method of claim 1, wherein the solid support comprises a plurality of interdigitated plates forming a flow channel, wherein at least some of the surfaces of the plates comprise capture probes, and wherein contacting the sample with a surface of a solid support comprises flowing the sample through the flow channel.

18. The method of claim 17, wherein the surfaces of the plates comprise electrodes.

19. The method of claim 18, wherein the surfaces of alternating plates comprise capture probes.

20. The method of claim 1, wherein the hybridization probe further comprises a third region adjacent the first region and opposite the second region, wherein the third region does not hybridize to the target nucleic acid.

21. A method for detecting a target nucleic in a sample, the method comprising:
melting the sample by heating the sample to a first temperature, wherein the sample comprises:
a primer which hybridizes to at least a portion of the target nucleic acid;
a hybridization probe comprising first and second regions, wherein the first region hybridizes to at least a portion of the target nucleic acid and the second region does not hybridize to the target nucleic acid and wherein the second region comprises a detectable label; and
a polymerase and an enzyme comprising nuclease activity wherein the polymerase extends the hybridized primer in the direction of the hybridized probe and the nuclease activity of the enzyme cleaves the hybridized probe to thereby release a probe fragment comprising the second region of the probe and the detectable label; and
and wherein the first temperature is above the melting temperature (Tₘ) of the primer and double stranded nucleic acids present in the sample that comprise the target nucleic acid;
subsequently annealing the sample by reducing the temperature to a second temperature lower than the first temperature to allow the primer and the hybridization probe to each hybridize to a single stranded portion of the target nucleic acid in the sample; and
subsequently elongating the primer by allowing the polymerase to extend the primer hybridized to the target nucleic acid at a third temperature thereby releasing the probe fragment;
optionally repeating melting, annealing and elongating at least once;
contacting the sample with a surface of a solid support, wherein the surface of the solid support comprises one or more capture probes which hybridize to at least a portion of the second region of the probe fragment;
allowing the capture probes to hybridize to at least a portion of the probe fragment in the sample to form a probe fragment/capture probe complex at a fourth temperature lower than the second and third temperatures; and
detecting label on the surface of the solid support;
wherein the capture probe more readily binds to the probe fragment than to the intact hybridization probe and wherein the hybridization probe is substantially single stranded at the Tₘ of the probe fragment/capture probe complex.

22. The method of claim 21, wherein the capture probe has a discrimination ratio of 3 or greater.

23. The method of claim 21, wherein the capture probe has a discrimination ratio of 5 or greater.

24. The method of claim 21, wherein the second temperature and the third temperature are the same.

25. The method of claim 21, wherein the polymerase and the enzyme comprising the nuclease activity are the same molecule.

26. The method of claim 25, wherein the polymerase is a thermostable enzyme.

27. The method of claim 26, wherein the thermostable enzyme is Taq polymerase.

28. The method of claim 21, wherein the surface of the solid support comprises an electrode and wherein the detectable label is a moiety that can transfer electrons to or from the electrode.

29. The method of claim 28, wherein the detectable label is a Ferrocene moiety.

30. The method of claim 28, wherein the surface of the solid support comprises gold.

31. The method of claim 21, wherein the solid support comprises a plurality of interdigitated plates forming a flow channel, wherein at least some of the surfaces of the plates comprise capture probes, and wherein contacting the sample with a surface of a solid support comprises flowing the sample through the flow channel.

32. The method of claim 31, wherein the surfaces of the plates comprise electrodes.

33. The method of claim 32, wherein the surfaces of alternating plates comprise capture probes.

34. The method of claim 21, wherein melting, annealing and elongating are performed multiple times in a series of cycles.

35. The method of claim 34, wherein detecting label on the surface of the solid support occurs after the last melting, annealing and elongating cycle.

36. The method of claim 21, wherein the sample is in contact with the surface of the solid support during melting, annealing and elongating and wherein detecting occurs multiple times during the method and/or after the last melting, annealing and elongation cycle.

37. A kit for detecting a target nucleic acid in a sample comprising:
a hybridization probe comprising a first region which hybridizes to at least a portion of the target nucleic acid and a second region comprising a detectable label wherein the second region does not hybridize to the target nucleic acid and wherein an enzyme comprising nuclease activity can cleave the hybridization probe when hybridized to the target nucleic acid to thereby produce a probe fragment comprising the second region and the detectable label;
a solid support comprising one or more capture probes on a surface thereof, wherein the capture probe hybridizes to at least a portion of the second region of the probe fragment to form a probe fragment/capture probe complex and wherein the capture probe more readily binds to the probe fragment than to the intact hybridization probe and wherein the hybridization probe is substantially single stranded at the Tₘ of the probe fragment/capture probe complex;
optionally, a primer which hybridizes to at least a portion of the target nucleic acid; and
optionally, a polymerase which extends the hybridized primer in the direction of the hybridized probe and an enzyme comprising nuclease activity to thereby cleave the hybridized hybridization probe and release of the probe fragment comprising the second region of the probe and the detectable label.

38. The kit of claim 37, wherein the capture probe has a discrimination ratio of 3 or greater.

39. The kit of claim 37, wherein the capture probe has a discrimination ratio of 5 or greater.

40. The kit of claim 37, wherein the surface of the solid support comprises an electrode and wherein the detectable label is a moiety that can transfer electrons to or from the electrode.

41. The kit of claim 40, wherein the detectable label is an electroactive Ferrocene moiety.

42. The kit of claim 40, wherein the surface of the solid support comprises gold.

43. The kit of claim 37, wherein the polymerase and the enzyme comprising nuclease activity are the same molecule.

44. The method of claim 1, wherein the nuclease activity is exonuclease activity.

45. The method of claim 21, wherein the nuclease activity is exonuclease activity.

46. The kit of claim 37, wherein the nuclease activity is exonuclease activity.
